(19) United States
(12) Patent Application Publication
(10) Pub. No.: US 2024/0157101 A1
(43) Pub. Date: May 16, 2024

(54) DELIVERY OF MACROMOLECULES USING MICROINJECTORS

(71) Applicant: THE JOHNS HOPKINS UNIVERSITY, Baltimore, MD (US)

(72) Inventors: David H. Gracias, Baltimore, MD (US); Florin M. Selaru, Baltimore, MD (US); Arijit Ghosh, Baltimore, MD (US)

(21) Appl. No.: 18/550,371

(22) PCT Filed: Mar. 18, 2022
(86) PCT No.: PCT/US22/20932

§ 371 Date: Sep. 13, 2023

Related U.S. Application Data
(60) Provisional application No. 63/163,094, filed on Mar. 19, 2021.

(51) Int. Cl. A61M 31/00; A61K 9/00; A61K 47/36

(52) U.S. Cl. CPC A61M 31/002; A61K 9/0031; A61K 9/0053; A61K 47/36

(57) ABSTRACT

The present disclosure provides microscale devices, systems, and methods thereof for the delivery of therapeutic and prophylactic active agents (e.g., macromolecules).

### CROSS-REFERENCE TO RELATED APPLICATIONS

This application claims the benefit of U.S. Provisional Application Nos. 63/163,094, filed Mar. 19, 2021, the content of which is herein incorporated by reference in its entirety.

### STATEMENT REGARDING FEDERALLY SPONSORED RESEARCH OR DEVELOPMENT

This invention was made with government support under grant 2R01EB017742-05 awarded by the National Institutes of Health. The government has certain rights in the invention.

### TECHNICAL FIELD

The present disclosure relates to devices, systems, and methods for the delivery of therapeutic and prophylactic agents (e.g., macromolecules).

### BACKGROUND OF THE INVENTION

Oral delivery of macromolecules (molecular weight >900 Da) is predicted to improve treatment outcomes by reducing costs and increasing compliance. Oral delivery of many macromolecules like proteins is not possible because of degradation by the acid and digestive enzymes in the upper GI tract and limited absorption of large molecule drugs across the tight epithelial cell junctions. Over the past 100 years, researchers have used chemical modifications of the drug molecules to improve the diffusion of macromolecular drugs or encapsulate them in permeation enhancing particles like chitosan nanoparticles or dendrimers and microneedle patches, but only with very limited success. For example, one of the most vexing issues in the field is the oral delivery of insulin, which has still proven challenging even with significantly innovative devices. The delivery of anti TNF-α drugs like infliximab poses even a bigger challenge because these molecules are more than 30 times larger than insulin, which makes the absorption impossible and oral ingestion of these molecules results in negligible bioavailability.

Over the last few years there has been a paradigm shift in the approach, where instead of using chemical modifications, researchers are trying to come up with ingestible self-injecting tools for the GI tract, which can inject macromolecular drugs across the epithelial cell junctions. However, currently there is no FDA (Food and Drug Administration) approved method to achieve intraluminal injection and it is difficult to achieve intraluminal injection using devices which are small enough to not cause any blockage in the GI tract.

### SUMMARY OF THE INVENTION

The present invention is directed to microinjection devices comprising an autonomous actuator and one or more microinjectors operably connected to the autonomous actuator, wherein at least a portion of the one or more microinjectors comprises an active agent. In some embodiments, the device is less than 1 cm. In some embodiments, the device is configured to be delivered via the gastrointestinal tract, the urinogenital system, respiratory tract, mammary ducts, or bile ducts.

In some embodiments, the active agent is on a tip of the one or more microinjectors (e.g., in a patch or reservoir on the tip). In some embodiments, at least a portion of the one or more microinjectors (e.g., the patch on the tip of the microinjectors) further comprises chitosan, alginate, poly(lacto-co-glycolic acid), polycaprolactone, acrylamide, paraffin wax or a combination thereof. In some embodiments, the active agent comprises a pharmaceutical agent, a macromolecule, or a combination thereof. In select embodiments, the active agent is selected from the group consisting of: insulin, infliximab, adalimumab, certolizumab, and vancomycin.

In some embodiments, the device further comprises a protective coating. In some embodiments, the protective coating is an enteric coating. In some embodiments, the protective coating comprises poly-methacrylate, alginate, methylcellulose, or a combination thereof.

The present invention is also directed to systems comprising a plurality of the devices described herein and a shell enclosing the plurality of devices or a scaffold supporting the plurality of devices on an outer surface.

In some embodiments, the shell is a capsule. In some embodiments, the shell comprises an enteric coating. In some embodiments, the shell comprises a first chamber comprising at least one aperture covered by the enteric coating and a second chamber comprising the microinjection devices. In some embodiments, the first chamber further comprises a composition configured to produce a gas upon contact with a fluid and the second chamber further comprises: a cap; a channel configured to transfer the gas from the first chamber; and a piston configured to remove the cap and release the microinjection devices. In some embodiments, the first chamber comprises a compressed spring having a pH response trigger operably connected to a plunger and the second chamber comprises a one-way valve configured to release the microinjection devices upon triggering of the compressed spring.

In some embodiments, the scaffold comprises a biodegradable polymer, a biomaterial, or a combination thereof.

The present invention further discloses methods of treating or preventing a disease or disorder in a subject comprising administering one or more of the devices or the systems to the subject. In some embodiments, the administration is orally or rectally. In some embodiments, the administration is to the urinogenital system, respiratory tract, mammary ducts, or bile ducts. In some embodiments, the administration is by endoscopy.

The present invention additionally discloses methods for fabricating one or more microinjection devices or the systems disclosed herein. The methods may comprise at least one or all of: depositing a trigger material and a stressed material on a substrate; patterning the active agent on microinjector tips; and applying a protective coating to the one or more microinjection devices. The methods may further comprise releasing the one or more devices from the substrate or wrapping the substrate with the one or more microinjection devices on an outer surface of a three-dimensional scaffold.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic of delivery of macromolecules across the GI mucosa by exemplary autonomous microinjectors.

FIG. 2 is a photograph of an exemplary microinjector on a fingertip (left), the extremely small size will not cause any intestinal blockage, and a bright field image (right) of four autonomous exemplary microinjectors after actuation in response to a temperature change. Scale bar=1 mm.

FIG. 3 is scanning electron microscope (SEM) images of an exemplary microinjector, where the tips are covered by a drug loaded patch of chitosan. Scale bar of the image on the left is 100 mm and the image on the right is 10 mm.

FIG. 4 is a graph of the cumulative release of a macromolecular drug, human insulin, in phosphate buffered saline from exemplary microinjectors over a duration of 6 hours. The data is normalized by the total number of injectors.

FIGS. 5A-5C are images showing the control of the porosity of the chitosan drug matrix electrodeposited on the microinjector tips. FIG. 5A is chitosan from shrimp shells. FIGS. 5B and 5C are medium molecular weight chitosan with 75% deacetylated (FIG. 5B) and 85-90% deacetylated (FIG. 5C).

FIG. 6A is an image of the penetration of exemplary multi-hinged injectors into tissue mimicking gelatin. Scale bar=100 μm. FIG. 6B is an image showing an exemplary microinjector where a model fluorescent drug is loaded on the tips of the microinjector.

FIGS. 7A-7D are images of the penetration of exemplary injectors of SIM into freshly excised rat intestinal mucosal tissue. 0.25 mm sized microinjector microcomputed tomography (μCT) (FIG. 7A) and SEM (FIG. 7B); 1.5 mm sized microinjector μCT (FIG. 7C) and SEM (FIG. 7D).

FIGS. 8A and 8B are images of the penetration of exemplary injectors in freshly excised pig mucosal tissue from the intestine and the stomach. FIG. 8A are SEM images. FIG. 8B is the μCT image.

FIG. 9 shows the rectal delivery and attachment of exemplary injectors to the rat colonic mucosa.

FIG. 10 is μCT (top) and SEM (bottom) images showing the penetration of exemplary injectors into the mucosa upon rectal administration in rats. Scale bars=100 μm.

FIG. 11 is a schematic of using exemplary dissolvable orally ingestible microinjectors. Devices capable of actively injecting a biologic across the intestinal mucosa may be housed in an enteric capsule. After a few hours, the injectors will dissolve away. Injectors are activated in the small intestine and deliver the drug.

FIG. 12 is a schematic of oral delivery of macromolecular drugs using exemplary ingestible multi-fingered injectors. The capsule is protected through the acidic stomach by an enteric polymer (green) and passes safely through the GI tract after delivering the drug, as shown on the left. The mechanism of drug delivery in which exemplary prestressed actuators are activated when the trigger material dissolves in intestinal fluid and delivers the drug by penetrating the intestinal epithelium as shown on the right.

FIG. 13 is an exemplary fabrication scheme of the exemplary multi-fingered microinjector module shown in FIG. 12. The fabrication is completed on a bioresorbable tape, on which two steps of sequential patterns are formed by stencil assisted deposition. In the first step, a magnesium sacrificial or trigger layer is deposited followed by the deposition of a bilayer of iron (Fe) and molybdenum (Mo), which acts as the dissolvable prestressed bilayer. This is followed by a stencil assisted deposition of an emulsion of PLGA 50:50 and the drug mixture, on the injector tips. After that the entire device is coated with an enteric polymer (e.g., Eudragit L100) for protection in the stomach. On drying of the enteric coating, the flexible smart actuator device is wrapped on a hollow cylinder made of polycaprolactone to obtain the final device to be used for oral delivery.

FIGS. 14A-14C are illustrations of an exemplary shell for delivering the microinjector devices described herein.

FIGS. 15A and 15B are illustrations of an exemplary shell for delivering the microinjector devices described herein before (FIG. 15A) and after (FIG. 15B) activation.

FIG. 16A is a conceptual illustration showing two exemplary microinjectors with their injection tips penetrating the mucosa as the injector are heated to physiological temperature, while the macromolecular drugs are transported past the mucosal epithelium. FIG. 16B is an image of four microinjectors as fabricated on a silicon wafer. FIG. 16C is a fluorescence image of an array of as fabricated microinjectors with a model drug rhodamine on the injection tips; (inset) zoomed in fluorescence image of a single microinjector.

FIGS. 17A-17C show penetration of in vitro tissue mimicking gels by microinjector tips. FIG. 17A is a confocal image showing the microinjection tips (loaded with rhodamine dye for ease of visualization) penetrating the 2 kPa gelatin (colored with 200 nm diameter fluorescent polystyrene beads). Scale bar is 100 μm. FIG. 17B is a cross sectional confocal fluorescence microscopy image of the injection tips penetrating a 2 kPa (top) and a 25 kPa (bottom) gelatin layer. The images clearly show that the microinjection tips penetrate much deeper into the soft gelatin. FIG. 17C is a graph of the depth of penetration of the microinjection tips into the gelatin of two different stiffnesses.

FIGS. 18A-18 show operation of the exemplary microinjector devices as described herein on ex vivo rat colon. FIG. 18A is an image of microinjectors before actuation on freshly excised rat colon tissue. Zoomed in image of a microinjector before (FIG. 18B) and after (FIG. 18C) actuation at the physiological temperature. FIG. 18D is a SEM image of a microinjector attached to the rat colon tissue. FIG. 18E is a SEM image showing the penetration of an injection tip into the colon tissue. FIG. 18F is a μ-CT examination of a rat colon with microinjectors attached to it. FIG. 18G is a zoomed in μ-CT image of the microinjector marked in panel f, showing the depth of penetration into the tissue. Scale bars of a-d are 1 mm. Scale bar of e is 50 μm. Scale bars of f and g are 1 mm.

FIGS. 19A-19E show enteral delivery of human insulin in live rats using exemplary microinjector devices described herein. FIG. 19A is a schematic showing the details of the experiment performed, which involves the intrarectal administration of human insulin formulated using microinjectors using controlled air pressure from a microfluidic controller (MFCS). FIG. 19B is a μ-CT image of rat colon, 48 hours post rectal administration, showing individual microinjectors (FIG. 19C). FIG. 19D is a graph showing the cumulative amount of human insulin released in saline from around 100 microinjectors, normalized by the number of injectors. Each microinjector can accommodate 300μ IU of human insulin on an average. The in vitro release experiments were conducted in an oven set at 37 deg C. FIG. 19E is a graph showing the comparison of the area under the PK curves for human insulin delivered by three different methods as mentioned in the main text. A significant advantage of using microinjectors, which can increase the total exposure by more than 3 times than reconstituted insulin, is apparent from the graph.

### DETAILED DESCRIPTION OF THE INVENTION

The present disclosure provides devices, and systems and methods thereof, for the delivery of therapeutic and prophylactic agents. The autonomous microscale injector devices and systems can perform transmucosal injection of macromolecular drugs (e.g., biologics) in the gastrointestinal (GI) tract. The microinjection devices and systems can deliver the drug in response to physiological cues like wettability, temperature, or pH. Each self-folding multi-injector device delivers enough force to penetrate the GI mucosa, thereby delivering the drug that is encapsulated on the injector tips closer to the underlying blood vasculature. The devices, and systems and methods thereof, may be applied to a wide variety of drugs, for example, insulin, anti-inflammatory macromolecules like infliximab, and the like. The method can be used with or as a substitution for current delivery methods of drugs, which predominantly includes intravenous infusions.

Herein, exemplary microinjectors autonomously delivered peptides across the GI epithelial tissue. The robotic microinjectors used energy stored in prestressed thin films to overcome the epithelial cell barrier in the colon and safely delivered insulin systemically. The microinjectors were able be used in large numbers inside a rat colon without causing any GI blockage or visible trauma in the animals.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, molecular biology, immunology, and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing,” are used interchangeably herein and refer to the placement of the devices of the disclosure into a subject by a method or route which results in at least partial localization a desired site. The devices can be administered by any appropriate route which results in delivery to a desired location in the subject.

As used herein, the term “biologic” or “biopharmaceutical” is a product (e.g., polypeptides or proteins, nucleic acids, viruses, or vaccines) manufactured in, extracted from, or synthesized, wholly or at least in part, from biological sources, or chemically synthesized de novo to mimic their naturally produced counterparts. Biologics can comprise sugars, proteins, nucleic acids, or combinations of the substances. Often, the biologics are isolated from human, animal, plant, fungal or microbial cells. Biologics may be substances identical or nearly identical to a natural product (e.g., erythropoietin, growth hormones, biosynthetic human insulin, and its analogues), antibodies that are custom-designed to counteract or block a given substance or target a certain cell type, or fusion proteins (e.g., a naturally occurring receptor linked to the immunoglobulin frame.

A “biomarker” includes a biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, and a small molecule, that is present in the biological sample and that may be isolated from, or measured in, the biological sample (e.g., tissues, fluids, and cells). Furthermore, a biomarker may be the entire intact molecule, or a portion thereof that may be partially functional or recognized, for example, by an antibody or other specific binding protein. A biomarker may be associated with a given state of a subject, such as a particular stage of disease (e.g., a cancer biomarker).

As used herein, a “nucleic acid” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. The term “nucleic acid” may also encompass a chain comprising non-natural nucleotides, modified nucleotides, and/or non-nucleotide building blocks that can exhibit the same function as natural nucleotides (e.g., “nucleotide analogs”). Further, the term “nucleic acid” as used herein refers to an oligonucleotide, nucleotide or polynucleotide, and fragments or portions thereof, and to DNA or RNA of genomic or synthetic origin, which may be single or double-stranded, and represent the sense or antisense strand.

A “polypeptide,” “protein,” or “peptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide,” “protein,” and “peptide” are used interchangeably herein.

The term “antibody,” as used herein, refers to a protein that is endogenously used by the immune system to identify and neutralize foreign objects, such as bacteria and viruses. Typically, an antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2, and CH3) regions, and each light chain contains one N-terminal variable (VL) region and one C-terminal constant (CL) region. The light chains of antibodies can be assigned to one of two distinct types, either kappa (u) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other. The variable regions of each pair of light and heavy chains form the antigen binding site of an antibody. The VH and VL regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)). The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the invention. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CH1 domains, (ii) a F(ab′)2 fragment, which is a bivalent fragment comprising two Fab fragments linked by a disulfide bridge at the hinge region, (iii) a Fv fragment consisting of the VL and VH domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (VH or VL) polypeptide that specifically binds antigen.

As used herein, the term “preventing” refers to partially or completely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, “treat,” “treating,” and the like means a slowing, stopping, or reversing of progression of a disease or disorder. The term also means a reversing of the progression of such a disease or disorder. As such, “treating” means an application or administration of the methods or devices described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of devices and systems contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Microinjection Devices

The present disclosure provides microinjection devices, systems, and methods of fabricating thereof.

a. Microinjection Devices

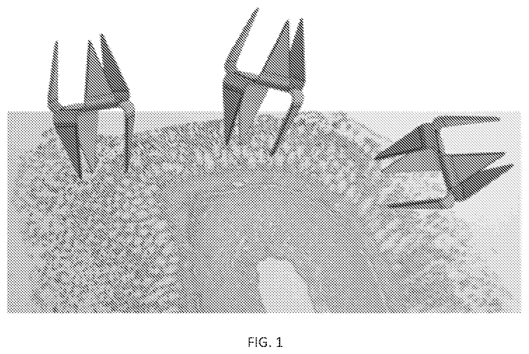

The microinjection devices comprise an autonomous actuator and one or more microinjectors operably connected to the autonomous actuator, wherein at least a portion of the one or more microinjectors comprises an active agent. In some embodiments, each of the one or more microinjectors may be operably connected to a single autonomous actuator. Alternatively, in some embodiments, a portion or all of the of the one or more microinjectors are operably connected to different autonomous actuators. Thus, the device may comprise one or more autonomous actuators with one or more operably connected microinjectors. As shown in FIGS. 1-3, the microinjectors may extend from the same or opposing faces of the device and may encircle a central hub of the device.

The autonomous actuator facilitates a structural configuration change in the device which in turn allows the microinjectors, and thus the active agent, to penetrate a tissue.

The autonomous actuator may comprise a stressed material and a trigger material. The stressed material refers to a material that stores torsional energy, as a result of inherent or intrinsic mechanical stress, such that release of torsional energy causes a change in structural configuration of the device. A typical strategy in producing a layer or film of a stressed material is to induce such stress during formation, such as by vapor deposition, of the material. If the stressed material is a stressed metal, either a tensile stress or a compressive stress, or a stress gradient can be imparted to a layer of the metal by appropriate selection of the deposition conditions. In one aspect of the exemplary embodiment, the stressed metal layer is formed by, in a controlled manner, depositing the material with compressive or tensile stress or a stress gradient in the layer. As will be appreciated, the force generated during the actuation process can be tuned by changing the composition and orientations of the stressed materials. When the actuator releases the torsion stress the force may be from several nanonewtons to millinewtons.

Exemplary stressed materials include, but are not limited to, metals, alloys, oxides, nitrides, semiconductors, Si, carbide, diamond, and various polymers etc. which have controllable internal stress or stress gradients. Representative stressed materials include, but are not limited to, metals and alloys such as molybdenum, tungsten (W), titanium-tungsten alloy (TiW), chromium (Cr), molybdenum-chromium alloy (MoCr), nickel (Ni), and nickel-zirconium alloy (NiZr). In some embodiments, the stressed material comprises iron, molybdenum, chromium, copper, gold, or combinations or alloys thereof.

A collection of stressed material layers can be utilized instead of a single layer of a stressed material. In the case of stressed metals, it may be advantageous to use two or more layers in order to achieve a predetermined desired total stress for the layered system. For example, a desired total tensile stress can be obtained by the use of two layers of a stressed material if both stressed material layers each have a tensile stress which sums to the desired total, or if one has a compressive stress, then the other must have a tensile stress greater than the magnitude of the compressive stress of the other, and the respective stresses must sum to the desired total. The layered assemblies which use multiple layers of stressed material films can use two or more layers of the same type of stressed material, two or more layers of the same type of stressed material however having different characteristics, or two or more layers of different types of stressed materials. In some embodiments, the stressed material comprises single- or multi-layered thin metal films. The actuator may include layers of other neutral or rigid materials in addition to the stressed material.

The trigger material is adjacent to the stressed material and is configured to prevent the change in structural configuration until a change in local conditions permits release of torsional energy. The change in local conditions may include a change in temperature, wettability, or pH. In some embodiments, the change in local conditions is a change in temperature. The change in local conditions may also include presence of a chemical and/or biological agent in the desired location for activation. The chemical or biological agent may comprise a biomarker, a protease, or an enzyme. The change in local conditions may result in etching, dissolving, or in some way weakening the trigger material such that it can no longer prevent the change in structural configuration.

In some embodiments, the trigger material responds to temperature, including, without limitation, paraffin wax, carnauba wax, beeswax, lanolin and other long chain hydrocarbons and complexes thereof. In some embodiments, the trigger material responds to local pH, including, without limitation, cellulose, gelatin, and the like. In some embodiments, the trigger material responds to dissolution in aqueous medium, including, without limitation, water soluble polymers, for example, polyethylene oxides, acrylates and polyacrylates, acrylamides and polyacrylamides, dextran, poly(methylacrylic) acid, polystyrene sulfonate, chitosan, alginate, metals and metal oxides for example, magnesium, magnesium oxide, zin oxide, zinc, iron, iron oxide, molybdenum. In some embodiments, the trigger material comprises single or multi-layered metal films. In exemplary embodiments, the trigger material comprises magnesium oxide.

Using multi-layered metal films, one of skill in the art can influence the directionality and magnitude of the resulting conformational change. For example, actuators with chromium below gold flex downwards while those with chromium on top of the gold flex upwards. Thus, the actuators flex in the direction of the layer which was under stress (e.g., the chromium layer as described above, denoted σ_{T}).

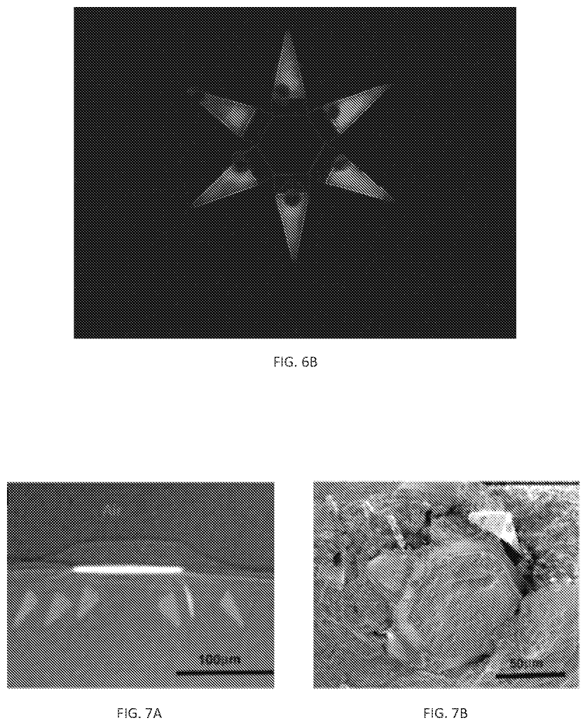

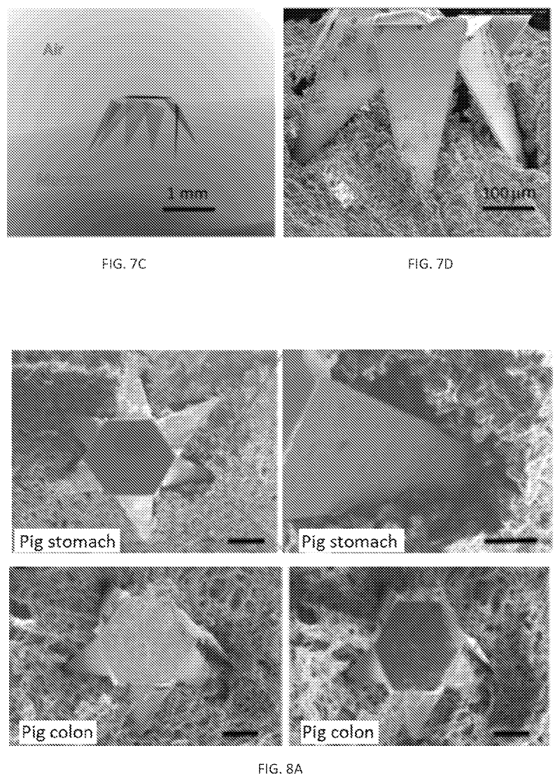

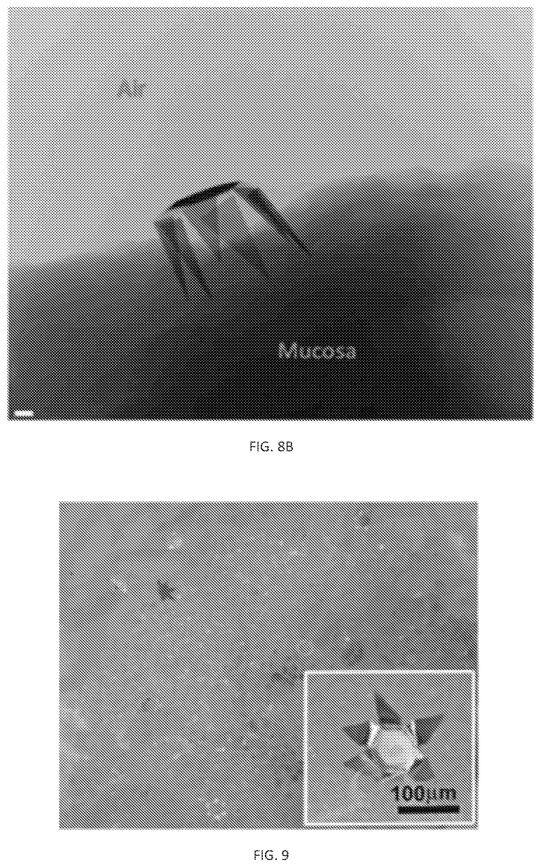

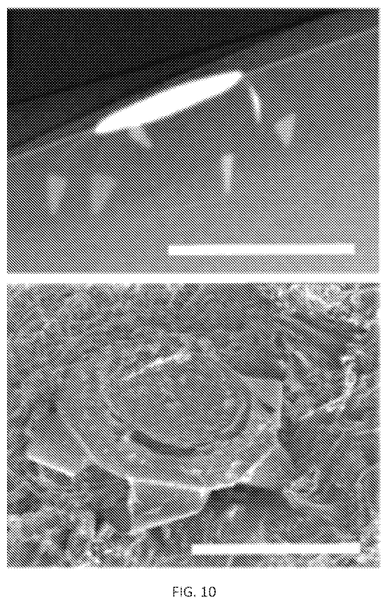

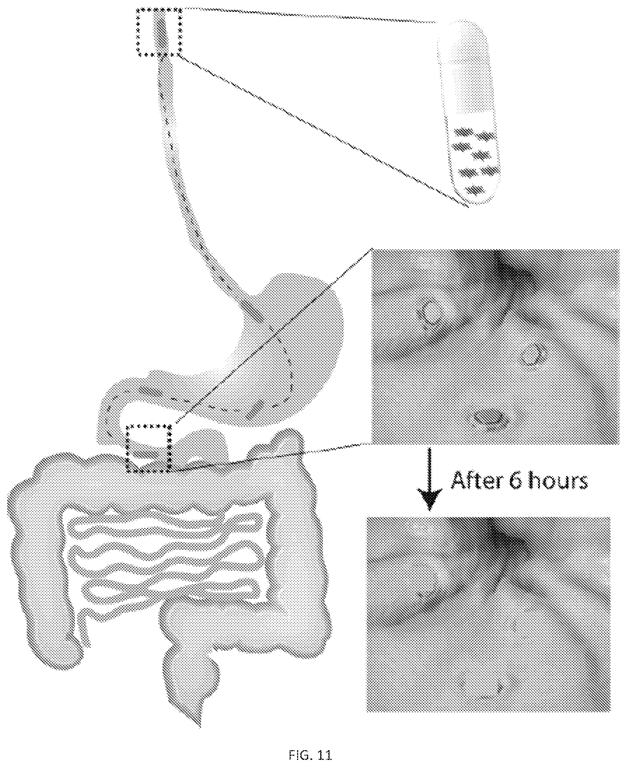

The change in structural configuration can include a change of one or more portions of the device with respect to other portions of the device, for example, by movement. Such movement may include, but is not limited to, rotational degrees of freedom, such as bending, pivoting, flexing, folding, rotating, twisting, or any combination thereof. This may include one or more sections configured as a hinge or to otherwise provide hinge-like motion. In some embodiments, the microinjectors may be operably connected to the autonomous actuator by a rigid or neutral material such that changes in the actuator configuration result in changes in orientation of the microinjectors with respect to the remainder of the device. The resulting structural configuration change thereby activates microinjectors to allow the microinjectors to penetrate a tissue as shown in FIGS. 6-10 and release the active agent (e.g., to the underlying bloodstream) as quantified in FIG. 4. For many active agents, the device increases the bioavailability of the drug by resulting in direct delivery to the bloodstream but without a systemic injection administration.

At least a portion or all of the one or more microinjectors comprise an active agent. In some embodiments, the active agent is on the tip of the one or more microinjectors.

The device is agnostic to the type of active agent. For example, any agent or agents suitable for delivery to a subject with the present device may be used. The active agent may comprise a pharmaceutical agent, a macromolecule, or a combination thereof. The macromolecule may be selected from the group consisting of: blood factors (e.g., Factor VIII, Factor IX); thrombolytic agents (e.g., tissue plasminogen activator); hormones (e.g., insulin, glucagon, growth hormone, gonadotrophins); hematopoietic growth factors (e.g., erythropoietin, colony-stimulating factors); interferons (e.g., interferons-α, -β, -γ); interleukin-based products (e.g., interleukin-2); vaccines (e.g., hepatitis B surface antigen); antibodies (e.g., monoclonal, polyclonal), and the like (e.g., tumor necrosis factor, therapeutic enzymes). In some embodiments, the active agent comprises a polypeptide, or a fragment thereof. In some embodiments, the active agent comprises an antibody, or a fragment thereof. In select embodiments, the active agent is selected from the group consisting of: insulin, exenatide, infliximab, adalimumab, certolizumab, vedolizumab, ustekinumab, evinacumab, ansuvimab, margetuximab, naxitamab, atoltivimab, maftivimab, tafasitamab, crizanlizumab, risankizumab, benralizumab, guselkumab, dupilumab, sarilumab, mepolizumab, nivolumab, pembrolizumab, ipilimumab, denosumab, tocilizumab, ofatumumab, eculizumab, ranibizumab, bevacizumab, trastuzumab, etanercept, belatacept, rilonacept, alefacept, abatacept, epoetin, interferons, filgrastim, growth hormones, coagulation factors, erythropoietin, gonadotropin, calcitonin, natriuretic peptides, human bone morphogenic proteins and combinations thereof.

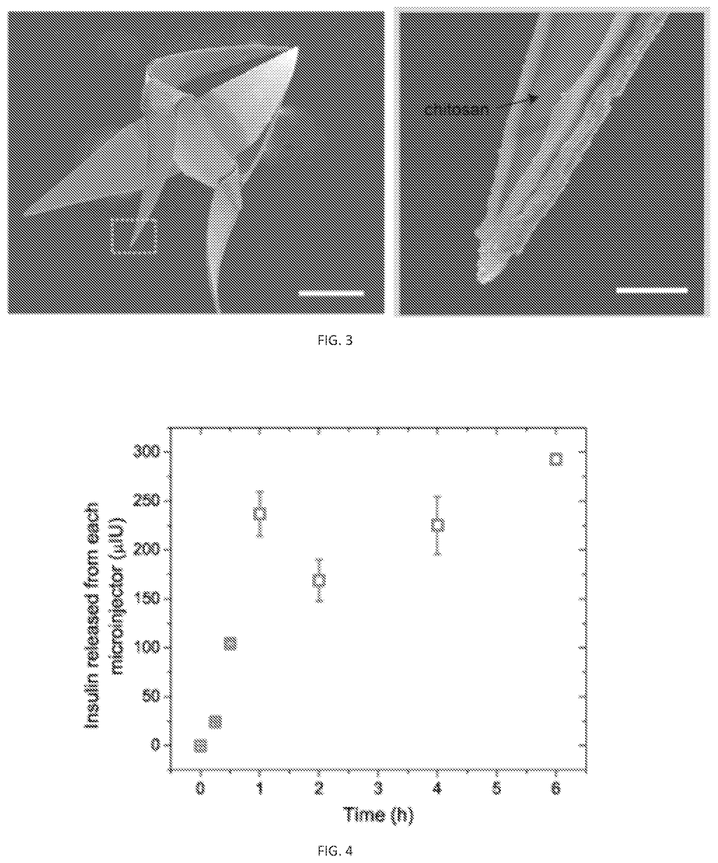

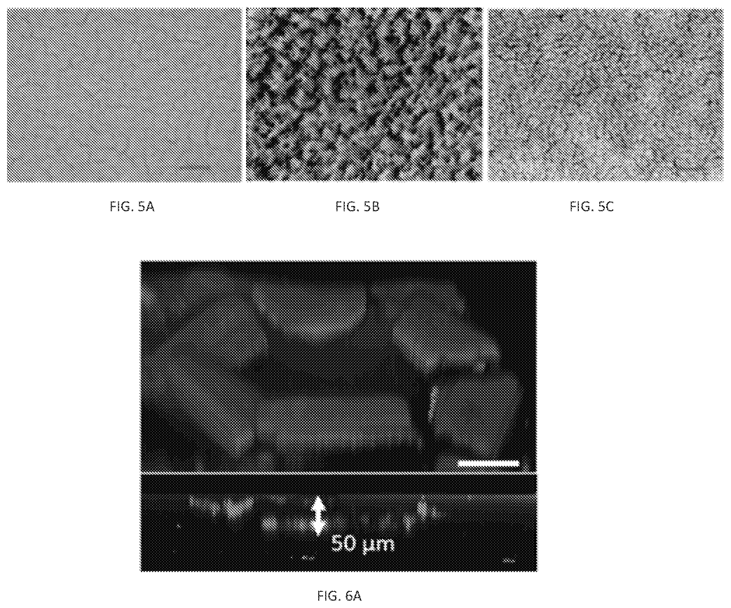

The active agent may be applied to the tip of the one or more microinjectors by a number of methods. In some embodiments, the active agent is supplied as a drug patch on the tip of the one or more microinjectors (FIG. 3). The drug patch or the tip of the microinjectors may comprise a biodegradable polymer, bioresorbable material, or another biomaterial. In some embodiments, the micro injector tip comprises chitosan, alginate, poly(lacto-co-glycolic acid), polycaprolactone, acrylamide, paraffin wax or a combination thereof. As will be appreciated, porosity and thickness of the drug patch can be controlled by methods of applying the particular materials and/or by using modified versions of the materials. For example, the porosity of a chitosan-based drug matrix or patch can be varied by using different molecular weights and chitosan species with different degrees of deacetylation of the chitosan, as shown in FIG. 5.

The tip of the microinjector can be any length or shape to facilitate injection of the active agent across the tissue of interest (e.g., intestinal mucosa) and into the blood stream. The tip may be 1 μm to 10 mm long. In some embodiments, the tip is 1 μm to 10 μm long, 1 μm to 100 μm long, 1 μm to 1 mm long, 10 μm to 100 μm long, 10 μm to 1 mm long, 10 μm to 10 mm long, 100 μm to 1 mm long, or 100 μm to 10 mm long. In some embodiments, the tip is flat. In some embodiments, the tip is cylindrical. In some embodiments, the tip is beveled. In some embodiments, the tip is serrated. In some embodiments, the tip is a hollow horizontal microneedle.

The microinjection device may further comprise a protective coating. The protective coating may include pH or time-dependent coatings, such that device is released in the vicinity of the desired application, or at various points and times, thus extending the duration of the delivery of an active agent. In some embodiments, the protective coating is an enteric coating. Commonly used enteric coatings include, but are not limited to, synthetic or modified natural polymers containing ionizable carboxylic groups. In the low pH environment of the stomach, the carboxylic groups remain un-ionized, and the polymer coatings remain insoluble. In the intestine, the pH increases to 5 and above, allowing the carboxylic groups on the polymeric coating materials to ionize, and the polymer coatings to disintegrate or dissolve. The protective coating may include, but is not limited to, one or more components selected from the group consisting of cellulose acetate phthalate, polyvinyl acetate phthalate, hydroxypropyl methyl cellulose phthalate, ethyl cellulose, poly-methacrylates or, specifically, EUDRAGIT coatings (available from Evonik Industries of Essen, Germany), waxes and shellac. In some embodiments, the protective coating comprises poly-methacrylate, alginate, methylcellulose, or a combination thereof.

The overall size of the microinjector device is dependent on the number of microinjectors and the length and shape of the microinjector tips. Different sizes and/or shapes of the devices can be provided depending on the specific application and site within a subject where they will be deployed. In some embodiments, the device may be less than 1 cm.

b. Systems

The systems may comprise a plurality of the devices disclosed herein and a shell substantially surrounding or enclosing the plurality of the devices. In some embodiments the shell is a capsule (e.g., for oral administration or as suppositories). The shell may comprise an enteric coating. The shell may include, but is not limited to, one or more components selected from the group consisting of cellulose acetate phthalate, polyvinyl acetate phthalate, hydroxypropyl methyl cellulose phthalate, ethyl cellulose, poly-methacrylates, or specifically EUDRAGIT coatings (available from Evonik Industries of Essen, Germany), waxes and shellac. In some embodiments, the shell comprises poly-methacrylate, alginate, methylcellulose, or a combination thereof.

As shown in FIG. 11, an exemplary system with a plurality of devices enclosed in an enteric capsule passes through the stomach following oral administration. Upon entry into the small intestine the enteric capsule solubilizes and the trigger material is activated allowing the exemplary microinjectors to deliver the active agent through the intestinal mucosa.

The shell may comprise a first chamber and a second chamber. In some embodiments, the first chamber comprises at least one aperture covered by the enteric coating. In some embodiments, the second chamber comprises the microinjection devices. The first chamber may further comprise a mechanism configured to interact with a biological fluid (e.g., fluid from the small intestine) which passes through the at least one aperture following dissolution of the enteric coating. The second chamber may further comprise a release mechanism to allow the microinjection devices to be released from the shell.

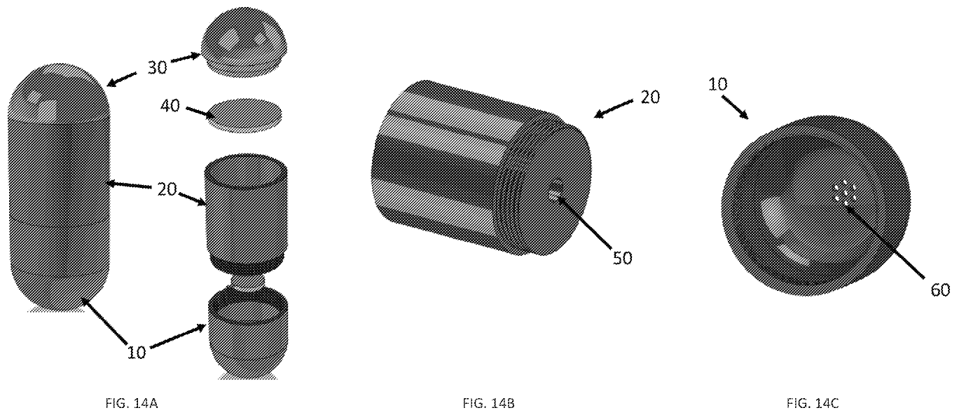

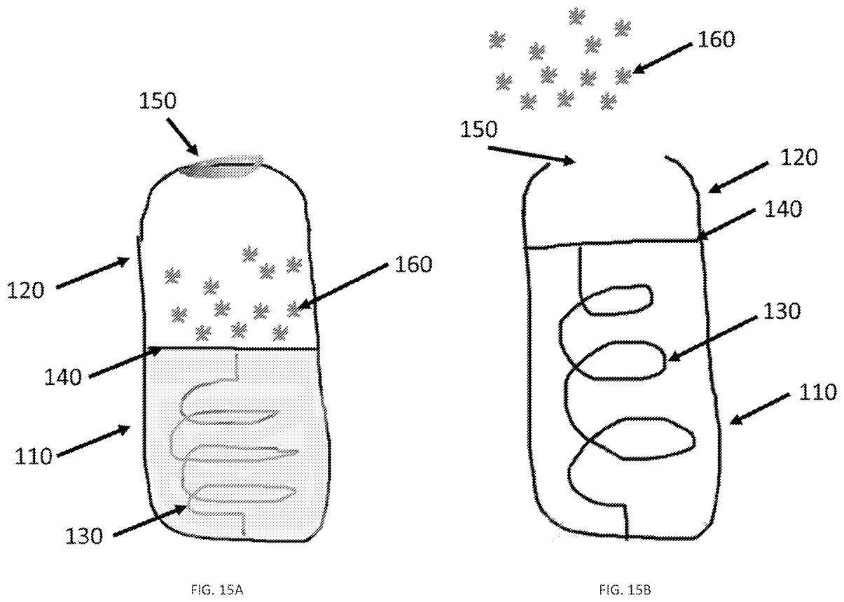

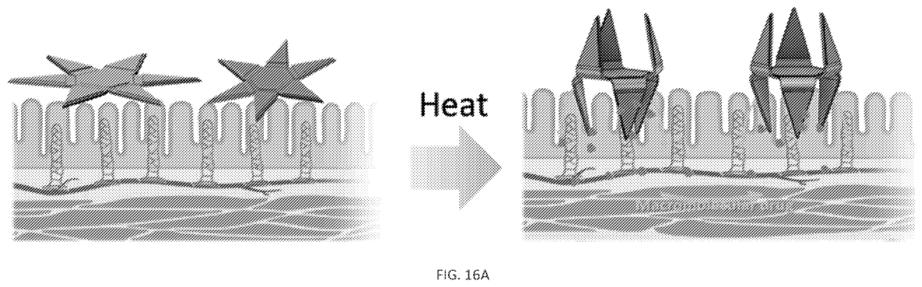

As shown in FIGS. 14A-14C, an exemplary shell has first chamber 10, second chamber 20, and cap 30 which may be removed by the action of piston 40. In some embodiments, first chamber 10 comprises a composition configured to produce a gas upon contact with a fluid and one or more apertures 60. For example, a mixture of citric acid and sodium bicarbonate or magnesium create CO₂ gas upon contact with aqueous fluids. In some embodiments, second chamber 20 comprises: cap 30; a channel 50 configured to transfer the gas from first chamber 10; and piston 40 configured to remove cap 30 and release the microinjection devices. Thus, the gas created in the first chamber passes through channel 50 and causes piston 40 to move and force off cap 30, thereby releasing the microinjection devices in the desired body cavity.

In some embodiments, as shown in FIG. 15A, first chamber 110 comprises pH responsive compressed spring 130 operably connected to plunger 140. The pH responsive compressed spring may comprise a pH responsive trigger material as described elsewhere herein. In some embodiments, second chamber 120 comprises one-way valve 150 configured to release microinjection devices 160 upon triggering of the compressed spring. One way valve 150 may be pressure sensitive such that movement of plunger 140 increases the pressure in second chamber 120 thereby opening the one-way valve 150 and releasing the microinjection devices 160 in the desired body cavity, as shown in FIG. 15B.

In some embodiments, the shell further comprises a desiccant. Desiccants are well known in the art and substances that absorb water or remove humidity, including, but not limited to, calcium chloride, calcium sulfate, magnesium chloride, magnesium sulfate, potassium carbonate, sodium chloride, sodium sulfate, sucrose, and the like. Herein, the desiccants may preserve any water soluble components within the device prior to activation and release.

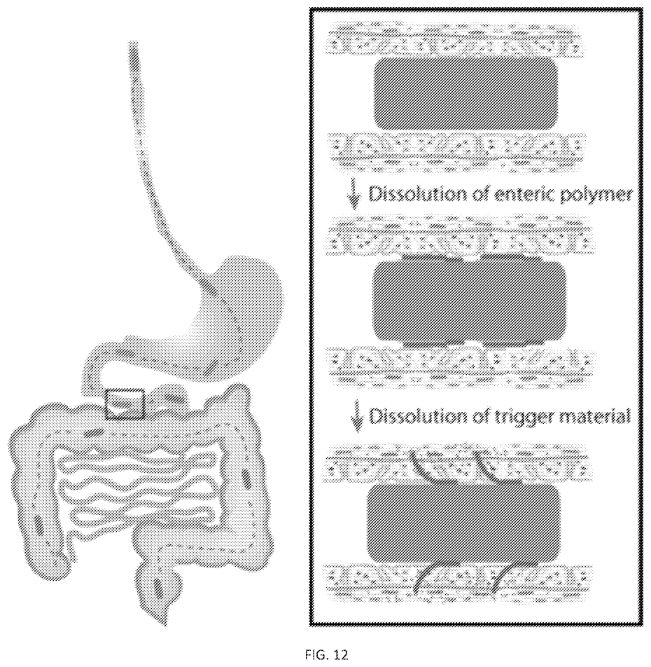

The systems may comprise a scaffold supporting a plurality of devices on its outer surface. The scaffold may be any size or shape configured to receive the one or more microinjection devices on the substrate. For example, the scaffold may be a cylinder or a sphere. The scaffold may comprise a biodegradable polymer or other implantable biomaterial. In some embodiments, the scaffold comprises polycaprolactone. FIG. 12 shows an exemplary system with a plurality of devices coated with an enteric coating on the outer surface of a cylindrical scaffold. After passing through the stomach following oral administration, entry into the small intestine solubilizes the enteric coating and activates the trigger material allowing exemplary microinjectors to deliver the active agent through the intestinal mucosa.

c. Methods of Fabrication

The devices and systems disclosed herein may be fabricated using various combinations of lithography (e.g., photolithography, stencil mask lithography), deposition (e.g., physical vapor deposition, electrodeposition), or coating (e.g., spin coating) techniques.

The methods may comprise at least one or all of depositing a trigger material and a stressed material on a flexible substrate; patterning the active agent on microinjector tips; and applying a protective coating to the one or more microinjection devices. In some embodiments, the active agent is provided as composition in chitosan, alginate, poly(lacto-co-glycolic acid), polycaprolactone, acrylamide, paraffin wax or a combination thereof. Alternatively, the methods may comprise depositing chitosan, alginate, poly(lacto-co-glycolic acid), polycaprolactone, acrylamide, paraffin wax or a combination thereof before or at the same time as the active agent.

In some embodiments, the substrate is flexible. In some embodiments, the substrate may be fully or partially releasable. Thus, in certain embodiments, the methods may further comprise releasing the one or more devices from the substrate. In some embodiments, the substrate comprises bioresorbable materials. Bioresorbable materials are generally polymeric materials which can be safely absorbed by the body so that the materials from which a construction is made disappear over time. The most common bioresorbable materials include polylactic acid (PLA), polyglycolic acid (PGA), poly-s-caprolactone (PCL) and derivatives (e.g., poly-L-lactide (PLLA), poly-D-lactide (PDLA) and poly-DL-lactide (PDLLA)) or copolymers (e.g., poly(lactic-co-glycolic acid) or PLGA, poly(glycolide-co-caprolactone) or PGCL, poly(glycolide-co-trimethylene carbonate) or (PGA-co-TMC)) thereof.

In some embodiments, the methods further comprise wrapping the substrate with the one or more microinjection devices on the outer surface of a three-dimensional scaffold. The scaffold may be any size or shape configured to receive the one or more microinjection devices on the substrate. For example, the scaffold may be a cylinder or a sphere. The scaffold may comprise a biodegradable polymer, bioresorbable material, as described above, or another implantable biomaterial. In some embodiments, the scaffold comprises polycaprolactone.

The methods may further comprise coating the one or more microinjection devices with a protective coating. The protective coatings described elsewhere herein are applicable and suitable for use with the disclosed methods.

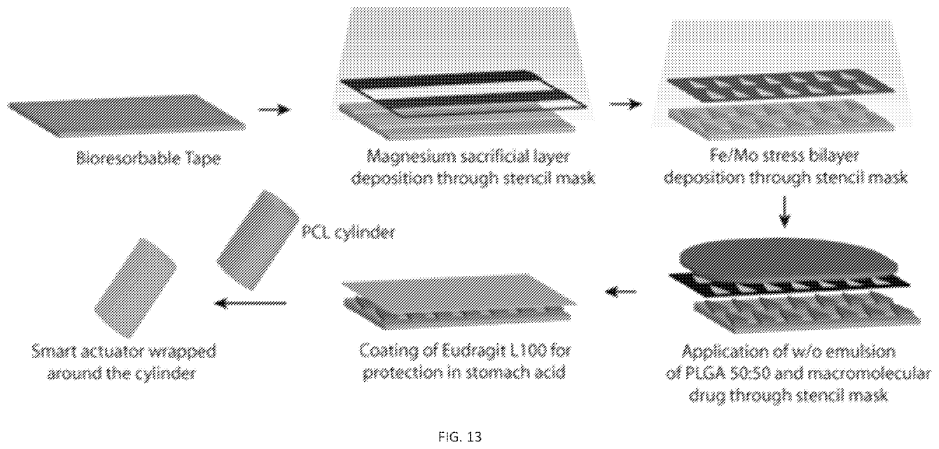

FIG. 13 shows an exemplary fabrication scheme of more than one microinjector devices disclosed herein. trigger (magnesium layer) and stressed (iron and molybdenum) materials are deposited on bioresorbable tape. An emulsion comprising PLGA and the active agent are deposited on top of the stressed material using stencil assisted deposition. All of the microinjector devices receive an enteric coating before the bioresorbable tape with the microinjector devices is wrapped on the outer surface of a cylindrical scaffold.

### 3. Methods of Treating or Preventing a Disease or Disorder

Also provided herein are methods of treating or preventing a disease or disorder comprising administering one or more of the disclosed devices or disclosed systems to a subject in need thereof.

In some embodiments, the administration is orally or rectally for example as a capsule or suppository or as a liquid (e.g., an enema). In some embodiments, the administration is to the urinogenital system (e.g., vaginally), respiratory tract (e.g., nasally), mammary ducts, or bile ducts. In certain embodiments, the administration is done by an endoscopy.

### 4. Examples

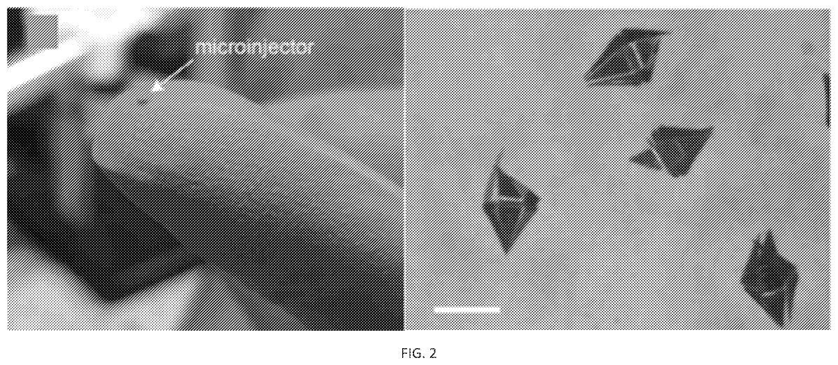

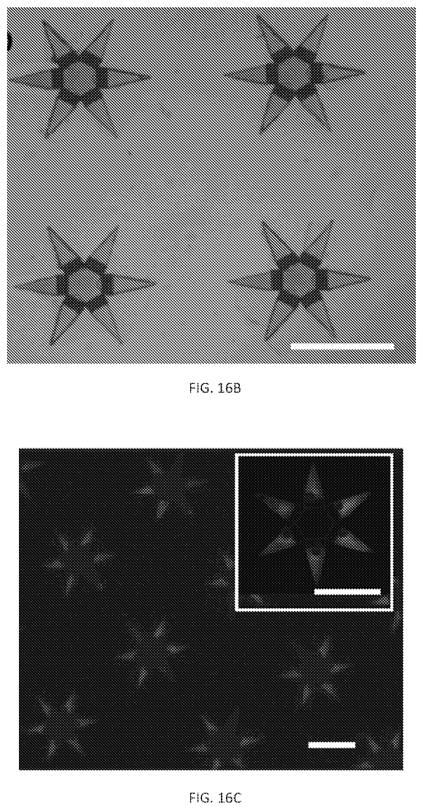

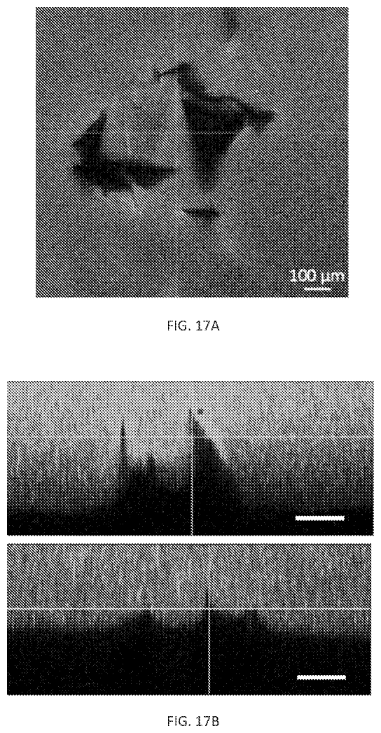

The microinjectors were designed using origami design principles, where different parts of the microinjector fold differently to generate the injection force on the tissue. The injection force is produced by controllably releasing the intrinsic stress in thin film assemblies of chromium (Cr) and gold (Au). Each microinjector was fabricated as a quasi-2D device 1.5 mm in tip-tip dimension, equipped with six injection tips, 450 μm in length (FIG. 16B). The injection tips, which contain a drug loaded polymer patch insert into the mucosal tissue during the process of 2D to 3D shape transformation of the microinjector (FIG. 2, right) and thus deliver the drug across the mucosal epithelial cells in the colon.

The design of the microinjectors allows the injection tips to deliver the drug in any direction, irrespective of the orientation of the flat device with respect to the surrounding colon tissue. This was achieved by carefully designing the folding directions of the microinjection tips, where alternate injection tips fold in opposite directions to each other. Two different stress layer assemblies were used for the opposite foldable microinjection tips: (i) a two-layer assembly of 60 nm Cr/100 nm Au and (ii) a four-layer assembly of 15 nm Cr/100 nm Au/75 nm Cr/5 nm Au. The choice of the layer thicknesses was based on the bending actuation geometries of multilayered thin films. The insulin loaded chitosan tips on the microinjectors was spatially patterned using a combination of photolithography and electrodeposition (FIG. 16C and inset). After fabrication, the microinjectors were released from the silicon wafer and stored at room temperature (23 deg C) or in a refrigerator prior to their characterization or use in insulin delivery experiments. FIG. 2, left, shows a single microinjector on a fingertip, in the pre actuated state, after its release from the silicon wafer.

The pressure exerted by the injection tips was calculated using Hertz contact mechanics model. The pressure exerted by the injection tips was significantly larger than the pressure exerted by the claws of similar microdevices, due to the larger hinge area in the microinjectors, which resulted in the generation of a much higher force. The injection tips were activated within a few minutes once the microinjectors reached the physiological temperature inside the GI tract, due to the softening of a paraffin wax thermoresponsive trigger which was patterned on the hinges of the injection tips. The insulin loaded chitosan patches on the six microtips of the microinjectors were less than 5 microns in thickness and did not reduce the sharpness of the microtips to a large extent (FIG. 3).

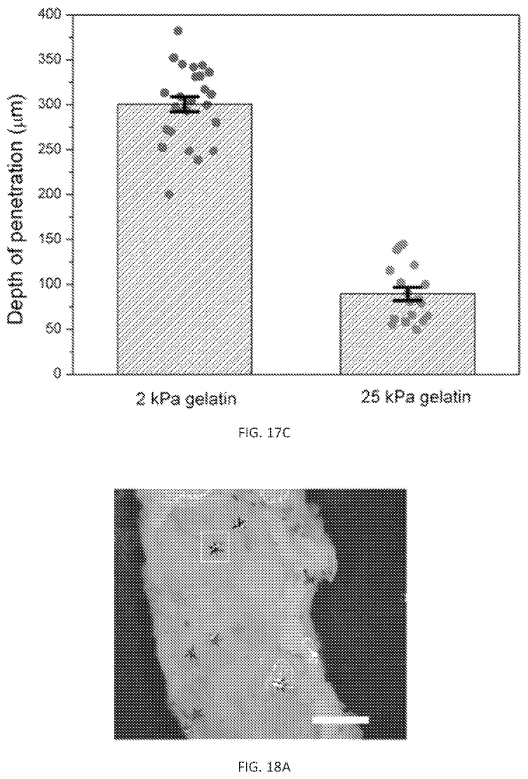

The injection performance of the microinjectors was evaluated on tissue mimicking gelatin having a stiffness of 1 to 2 kPa (FIG. 17A). For ease of visualization, a rhodamine dye was used as the model drug. The microinjection tips were then placed on the gel in an oven set at 40° C. for 15 to 20 minutes. The microinjectors were activated and inserted their injection tips into the gelatin. Similar experiments were carried out in gelatin of two different stiffnesses (FIG. 17B) and the microinjector tips could only penetrate up to 100 microns into the 25 kPa gel while it could penetrate up to 3 times the depth into 2 kPa gelatin (FIG. 17C).

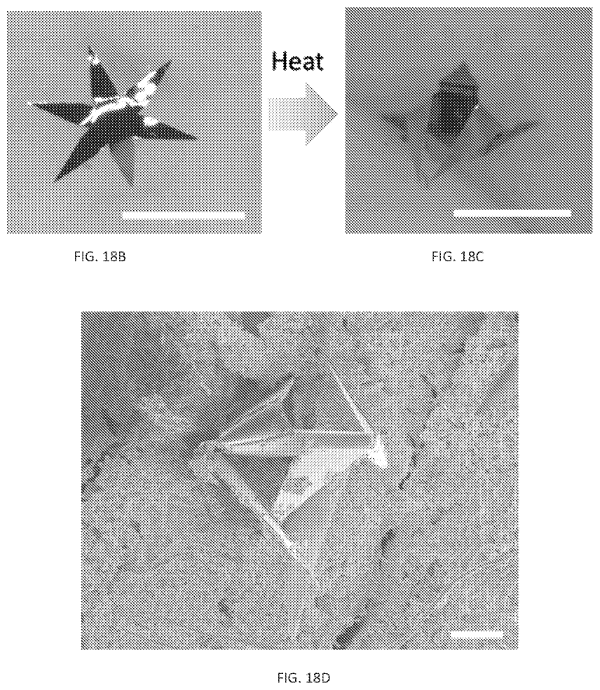

The performance of the microinjectors was also evaluated on ex vivo rat colon tissue. Freshly excised rat colon tissue was used and a few microinjectors were placed on the top of the tissue which was placed under saline in a petri dish (FIGS. 18A-18B). The tissue and injectors assembly were then heated in an oven to 40 deg C. (FIG. 18C). The tissue penetration abilities of the microinjectors were then evaluated by scanning electron microscopy (SEM) and micro computed tomography (μ-CT) examinations. As shown in FIGS. 18D-18G, the microinjectors could penetrate deep into the rat colon mucosal tissue to a depth of several hundred microns.

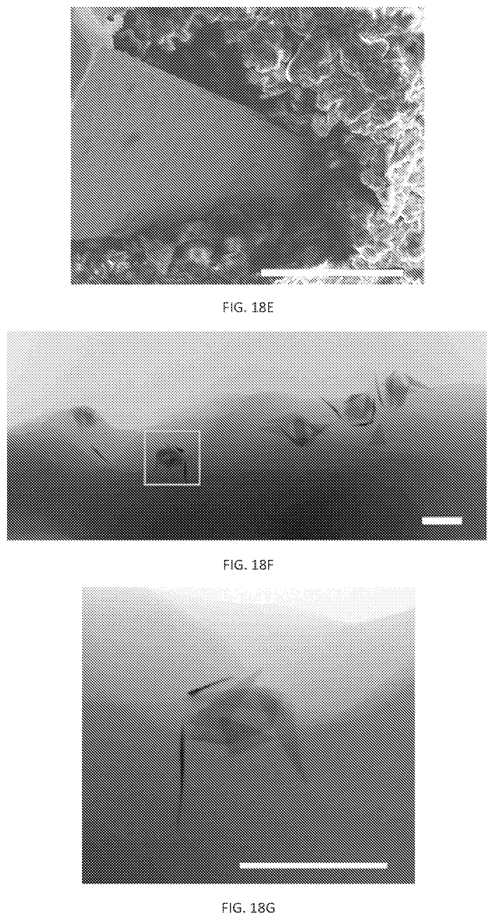

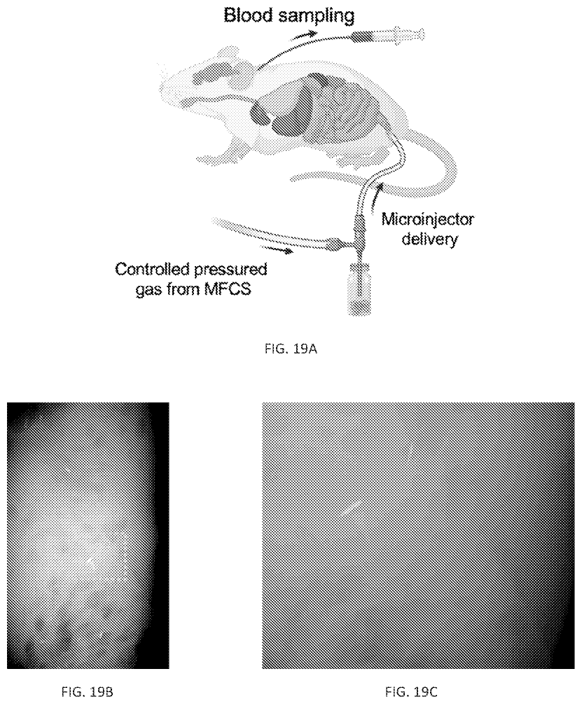

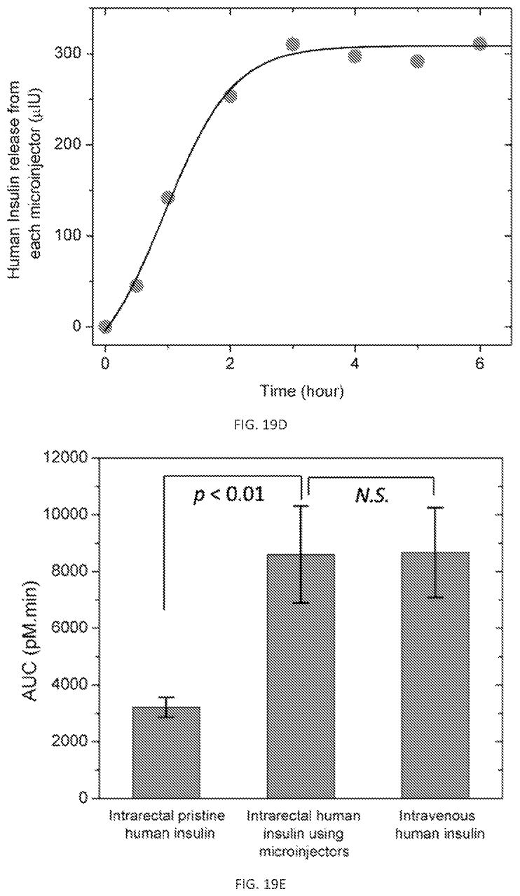

A large number of injectors (up to 200) were administered intrarectally into the colon of live rats (FIG. 19A). Wistar rats (280-350 g male) were used for the experiments and have a nominal colon diameter of 8 mm. The administration was accomplished by using a pneumatic microfluidic controller, which could eject a bolus of the microinjectors in saline using controlled pressure. A pressure of 13-16 psi with a medical grade polycarbonate tubing, of internal diameter 2.5 mm, was used to drive the bolus of microinjectors into the rat colon. No adverse effects were seen on the health of the animals over the duration of the experiments which was conducted for up to 48 hours post administration of the injectors. FIGS. 19B-19C show μ-CT images of microinjectors inside the colon of rats, 48 hours after their intrarectal administration.

Human insulin has been used in numerous studies in rats to develop methods for macromolecular drug delivery via the GI tract. Insulin was loaded into the microinjector tips by soaking them in a concentrated insulin solution and it was completely released over a duration of 3 to 4 hours from the microinjector tips (FIG. 19D). Each microinjector was found to accommodate around 300 mIU of human insulin. For insulin delivery experiments, 60 milli IU of human insulin was administered in each animal by three different means (N=5 for each group). The first animal group got an intrarectal dose of 60 milli IU of reconstituted human insulin in 1 ml of saline. The second group got an intrarectal dose of 60 milli IU of human insulin which is formulated into 200 microinjectors. The rectal administration for both these groups were carried out using the pneumatic controller. The third animal group got an intravenous (IV) dose of 60 milli IU of human insulin through a jugular vein catheter. Blood was drawn from all the animals at t=5 min, 30 min, 1 hr, 2 hr, 3 hr and 4 hr post administration of the insulin. An enzyme-linked immunosorbent assay (ELISA) was used to determine the concentration of human insulin at the various time points and measured the total exposure of insulin by calculating the area under the PK curve over the 4-hour time window. The microinjectors can deliver significantly higher amount of insulin to the bloodstream compared to the saline reconstituted insulin, and similar to the IV dosed animals (FIG. 19E).

Oral delivery of macromolecular drugs like insulin may dramatically improve patient outcomes and reduce costs by increasing compliance, decreasing complications, and hospitalizations. However, enhancing the diffusion of these drug molecules across the GI epithelium is challenging. The miniaturized self-injectors described herein are small enough to be safely ingested and can significantly enhance the absorption of molecules like insulin from the gastrointestinal tract.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A microinjection device comprising:
an autonomous actuator; and
one or more microinjectors operably connected to the autonomous actuator, wherein at least a portion of the one or more microinjectors comprises an active agent.

2. The microinjection device of claim 1, wherein each of the one or more microinjectors comprises a tip comprising hollow tapered microneedles or sharp or serrated tips.

3. The microinjection device of claim 1 or claim 2, wherein the active agent is on the tip of the one or more microinjectors.

4. The microinjection device of claim 3, wherein the active agent is in a patch on the tip of the one or more microinjectors.

5. The microinjection device of any of claims 1-4, wherein at least a portion of the one or more microinjectors further comprises chitosan, alginate, poly(lacto-co-glycolic acid), polycaprolactone, acrylamide, polyvinyl alcohol, polyvinylpyrrolidone, paraffin wax or a combination thereof.

6. The microinjection device of claim 5, wherein the portion is the patch on the tip of the one or more microinjectors.

7. The microinjection device of any of claims 1-6, wherein the active agent comprises a pharmaceutical agent, a macromolecule, or a combination thereof.

8. The microinjection device any of claims 1-7, wherein the active agent comprises polypeptide, or a fragment thereof.

9. The microinjection device of any of claims 1-8, wherein the active agent comprises an antibody or fragment thereof.

10. The microinjection device of any of claims 1-9, wherein the active agent is selected from the group consisting of; insulin, exenatide, infliximab, adalimumab, certolizumab, vedolizumab, ustekinumab, evinacumab, ansuvimab, margetuximab, naxitamab, atoltivimab, maftivimab, tafasitamab, crizanlizumab, risankizumab, benralizumab, guselkumab, dupilumab, sarilumab, mepolizumab, nivolumab, pembrolizumab, ipilimumab, denosumab, tocilizumab, ofatumumab, eculizumab, ranibizumab, bevacizumab, trastuzumab, etanercept, belatacept, rilonacept, alefacept, abatacept, epoetin, interferons, filgrastim, growth hormones, coagulation factors, erythropoietin, gonadotropin, calcitonin, natriuretic peptides, human bone morphogenic proteins, and combinations thereof.

11. The microinjection device of any of claims 2-10, wherein the tip of the one or more microinjectors is 1 micron to 10 mm long.

12. The microinjection device of any of claims 2-11, wherein the tip is flat, cylindrical, or beveled.

13. The microinjection device of any of claims 1-12, wherein the device further comprises a protective coating.

14. The microinjection device of claim 13, wherein the protective coating is an enteric coating.

15. The microinjection device of claim 13 or claim 14, wherein the protective coating comprises poly-methacrylate, alginate, methylcellulose, or a combination thereof.

16. The microinjection device of any of claims 1-15, wherein the device is less than about 1 cm.

17. The microinjection device of any of claims 1-16, wherein the device is configured to be delivered via the gastrointestinal tract, the urinogenital system, respiratory tract, mammary ducts, or bile ducts.

18. A system comprising:
a plurality of the microinjection devices of claims 1-17; and
a shell enclosing the plurality of devices or a scaffold supporting the plurality of devices on an outer surface.

19. The system of claim 18, wherein the shell is a capsule.

20. The system of claim 18 or claim 19, wherein the shell comprises an enteric coating.

21. The system of any of claims 18-20, wherein the shell comprises poly-methacrylate, alginate, methylcellulose, or a combination thereof.

22. The system of any of claims 18-21, wherein the shell comprises a first chamber comprising at least one aperture covered by an enteric coating and a second chamber comprising the microinjection devices.

23. The system of claim 22, wherein the first chamber further comprises a composition configured to produce a gas upon contact with a fluid.

24. The system of claim 23, wherein the second chamber further comprises:
a cap;
a channel configured to transfer the gas from the first chamber; and
a piston configured to remove the cap and release the microinjection devices.

25. The system of claim 22, wherein the first chamber comprises a compressed spring having a pH response trigger operably connected to a plunger.

26. The system of claim 25, wherein the second chamber comprises a one-way valve configured to release the microinjection devices upon triggering of the compressed spring.

27. The system of any of claims 19-26, wherein the shell further comprises a desiccant.

28. The system of claim 18, wherein the scaffold comprises a biodegradable polymer, a biomaterial, or a combination thereof.

29. A method of treating or preventing a disease or disorder in a subject comprising administering one or more of the devices of claims 1-17 or the system of claims 18-28 to the subject.

30. The method of claim 29, wherein the administration is orally or rectally.

31. The method of claim 29 or 30, wherein administration is to gastrointestinal tract, urinogenital system, respiratory tract, mammary ducts, or bile ducts.

32. The method of any of claims 29-31, wherein the administration is by endoscopy.

33. A method for fabricating one or more microinjection devices of any of claims 1-17 or the system of claims 18-28 comprising:
depositing a trigger material and a stressed material on a substrate;
patterning the active agent on microinjector tips; and
applying a protective coating to the one or more microinjection devices.

34. The method of claim 33, further comprising releasing the one or more devices from the substrate.

35. The method of claim 33 or 34, wherein the substrate is flexible.

36. The method of claim 35, further comprising wrapping the substrate with the one or more microinjection devices on an outer surface of a three-dimensional scaffold.

37. The method of any of claims 33-36, wherein the substrate comprises a bioresorbable material.

38. The method of any of claims 33-37, wherein the active agent is provided as a composition further comprising chitosan, alginate, poly(lacto-co-glycolic acid), polycaprolactone, acrylamide, paraffin wax or a combination thereof.

39. The method of any of claims 33-38, further comprising coating the one or more microinjection devices with a protective coating.

40. The method of claim 39, wherein the protective coating is an enteric coating.

41. The method of claim 39 or claim 40, wherein the protective coating comprises poly-methacrylate, alginate, methylcellulose, or a combination thereof.
